(19) United States
(12) Patent Application Publication
(10) Pub. No.: US 2008/0015671 A1
(43) Pub. Date: Jan. 17, 2008

(54) Method And Apparatus For Treatment Of Cardiac Valves

(76) Inventor: Philipp Bonhoeffer, London (GB)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/791,193

(22) PCT Filed: Nov. 21, 2005
(86) PCT No.: PCT/GB05/04469

§ 371 Date: May 18, 2007

Related U.S. Application Data
(60) Provisional application No. 60/629,438, filed on Nov. 19, 2004.

(51) Int. Cl. A61F 2/06

(52) U.S. Cl. 623/1.2

(57) ABSTRACT

Provided is a method and apparatus for placing a valve (14) in a tubular organ having a greater diameter than the valve, comprising: an expandable tubular adapter (10) having an outer portion with a diameter suitable for contacting the inner walls of the tubular organ, and an inner portion with a diameter suitable for placement of the valve; a valve mounted within the inner portion of the adapter; and a system for placing a valved vascular segment in a tubular organ having a greater inner diameter than the outer diameter of the vascular segment, comprising: an expandable tubular adapter having an outer portion with a diameter suitable for contacting the inner walls of the tubular organ, and an inner portion with a diameter suitable for placement of the valve; an expandable valved vascular segment, expandable to the diameter of the inner portion of the adapter.

### FIELD OF THE INVENTION

This invention relates generally to treatment of cardiac valve disease and more particularly to replacement of malfunctioning pulmonary valves.

### BACKGROUND OF THE INVENTION

Recently, there has been interest in minimally invasive and percutaneous replacement of cardiac valves. In the specific context of pulmonary valve replacement, US Patent Application Publication Nos. 2003/0199971 A1 and 2003/0199963 A1, both filed by Tower, et al. and incorporated herein by reference describe a valved segment of bovine jugular vein, mounted within an expandable stent, for use as a replacement pulmonary valve. The replacement valve is mounted on a balloon catheter and delivered percutaneously via the vascular system to the location of the failed pulmonary valve and expanded by the balloon to compress the native valve leaflets against the right ventricular outflow tract, anchoring and sealing the replacement valve. As described in the articles: “Percutaneous Insertion of the Pulmonary Valve”, Bonhoeffer, et al., Journal of the American College of Cardiology 2002; 39: 1664-1669 and “Transcatheter Replacement of a Bovine Valve in Pulmonary Position”, Bonhoeffer, et al., Circulation 2000; 102: 813-816, both incorporated herein by reference in their entireties, the replacement pulmonary valve may be implanted to replace native pulmonary valves or prosthetic pulmonary valves located in valved conduits.

While the approach to pulmonary valve replacement described in the above patent applications and articles appears to be a viable treatment, it is not available to all who might benefit from it due to the relatively narrow size range of available valved segments of bovine jugular veins. These venous segments are typically available only up to a diameter of about 22 mm. Unfortunately, the most common groups of patients requiring pulmonary valve replacement are adults and children who underwent transannular patch repair of tetralogy of Fallot during infancy. Their right ventricular outflow tracts are often larger in diameter.

Other implantables and implant delivery devices are disclosed in published U.S. Pat. Application No. 2003-0036791-A1 and European Patent Application No. 1 057 460-A1.

### SUMMARY OF THE INVENTION

The present invention is generally intended to provide a mechanism to allow the use of replacement valves in locations in which the diameter of the desired location of the replacement valve is greater than the diameter of the available replacement valve. More particularly, the invention is intended to provide a mechanism allowing use of valved segments of bovine jugular veins as replacement pulmonary valves in patients having large right ventricular outflow tracts. However, the invention may also be useful in conjunction with other replacement valves, for example as disclosed in. U.S. Pat. Nos. 6,719,789 and 5,480,424, issued to Cox.

The present invention accomplishes the above described objectives by providing an expandable adapter stent having a configuration which, when expanded, displays a larger diameter sections or sections having outer diameters sufficient to engage and seal against the inner wall of the vessel at the desired implant site and a reduced diameter internal section, having an inner diameter generally corresponding to the outer diameter of the valved venous segment or other replacement valve.

Thus, the present invention provides an apparatus for placing a valve in a tubular organ having a greater diameter than the valve, comprising:
an expandable tubular adapter having an outer portion with a diameter suitable for contacting the inner walls of the tubular organ, and an inner portion with a diameter suitable for placement of the valve;
a valve mounted within the inner portion of the adapter.

Thus, the expandable tubular adapter may be toroidal in form (see FIG. 11) having a smaller inner diameter portion and a larger outer diameter portion, or alternatively it may have a form approximate to a ‘dumbell’ (see FIG. 1 and FIG. 12). In this latter form, the adapter still comprises an outer portion suitable for contacting the vessel wall, and an inner portion for accepting the valve, but at some point toward the centre of the adapter the outer portion narrows in diameter to allow the adapter to sit within the vessel over the existing valves that the device is designed to replace.

It is particularly preferred that the adapter has a radial wall extending from the inner portion to the outer portion, so as to define a significant difference between the outer and inner diameter of the device. Thus, a single piece of woven wire (or a single thin layer of material) in a tubular or ‘dumbell’ shape, would not normally be sufficient to define sufficient difference between the outer and inner diameters of the adapter. The inner diameter is usually from 18-22 mm, whilst the outer diameter is from ≧22-50 mm, preferably ≧22-40 mm.

The material from which the adapter is made is not especially limited. However, it is particularly preferred that the material is flexible in order that it can form to the shape of the vessel within which is it implanted. This allows for a better seal with the vessel walls and also allows the device to flex with the vessel as it moves naturally within the body. It is also preferred that the outer portion of the adapter can be compressed to a certain degree, without significant compression of the inner portion. This allows the adapter to be subjected to normal stress and strain in the body, without constricting flow within the adapter. The flexible materials discussed herein are suitable for achieving this.

Particularly preferred materials include Nitinol, or other similar alloys, as explained below. The ends of the adapter (e.g. 104 and 106 on FIG. 11, 140 and 148 on FIG. 12) are preferably sealed to prevent leakage into the device, which can otherwise cause bypassing of the valve and lead to undesirable clotting. Suitable materials for this covering include collapsible materials, such as Gore-Tex®, or may also include valve tissue or venous tissue if desired.

The invention also provides a method for placing a valve in a tubular organ having a greater diameter than the valve, which method comprises:
delivering an expandable tubular adapter having an outer portion with a diameter suitable for contacting the inner walls of the tubular organ, and an inner portion with a diameter suitable for placement of the valve;
expanding the adapter so that the outer portion contacts the tubular organ; and
placing the valve within the inner portion of the adapter.

In this method, the valve may be placed in the adapter and then the adapter delivered to the organ if necessary. That is to say that the last step above may be performed first, if desired, since the order of the steps is not especially limited.

Thus, in one embodiment of the invention, the valved venous segment or other replacement valve is located in the internal section of the adapter stent prior to implant. In a second embodiment, the valved venous segment or other replacement valve is placed in the internal section of the adapter stent after previous implant of the adapter stent. In such an embodiment, the replacement valve may itself be mounted in an expandable valve stent, as described in the above cited Tower, et al., applications and Bonhoeffer, et al. articles. The stents employed in the invention may either be self-expanding stents, for example constructed of Nitinol or may be balloon expanded stents. In the preferred embodiments described below, the adapter stent is a self-expanding stent and the valve stent, if present, is a balloon expandable stent. In the preferred embodiments discussed below, the adapter stent is provided with a liquid resistant impermeable covering, e.g. ePTFE, polyurethane, or the like, so that blood flow is all directed through the replacement valve orifice.

In the preferred embodiments discussed below, the adapter stent is constructed of woven Nitinol wire, heat treated to memorize is configuration.

### BRIEF DESCRIPTION OF THE DRAWINGS

These and other advantages and features of the present invention will be appreciated as the same becomes better understood by reference to the following detailed description of the preferred embodiment of the invention when considered in connection with the accompanying drawings, in which like numbered reference numbers designate like parts throughout the figures thereof, and wherein:

FIG. 1 illustrates a side view of an adapter stent appropriate for use with all disclosed embodiments of the invention.

FIG. 2 illustrates an end view of the adapter stent of FIG. 1, with a valved venous segment installed, according to a first embodiment of the invention.

FIG. 3 illustrates a side view of the adapter stent of FIG. 1, provided with a liquid resistant covering.

FIG. 4 illustrates a delivery system for a replacement valve according to the first embodiment of the invention.

FIG. 5 illustrates a replacement valve according to the first embodiment of the invention as it is delivered by the system of FIG. 4.

FIG. 6 illustrates a functional cross section through a replacement valve according to the first embodiment of the invention, as implanted in the right ventricular outflow tract.

FIG. 7 illustrates a stented valved venous segment as described in the above cited Tower, et al. and Bonhoeffer, et al. references, for use in practicing the second embodiment of the invention.

FIG. 8 illustrates a delivery system for a delivering the valved venous segment of FIG. 7, for use in practicing the second embodiment of the invention.

FIG. 9 illustrates the valved venous segment of FIG. 7 as it is delivered by the system of FIG. 8.

FIG. 10 illustrates a functional cross sectional view through a replacement valve according to the second embodiment of the invention, as implanted in the right ventricular outflow tract.

FIG. 11 is a schematic drawing of a first alternative adapter stent appropriate for use with all disclosed embodiments of the invention.

FIG. 12 is a schematic drawing of a second alternative adapter stent appropriate for use with all disclosed embodiments of the invention.

### DETAILED DESCRIPTION

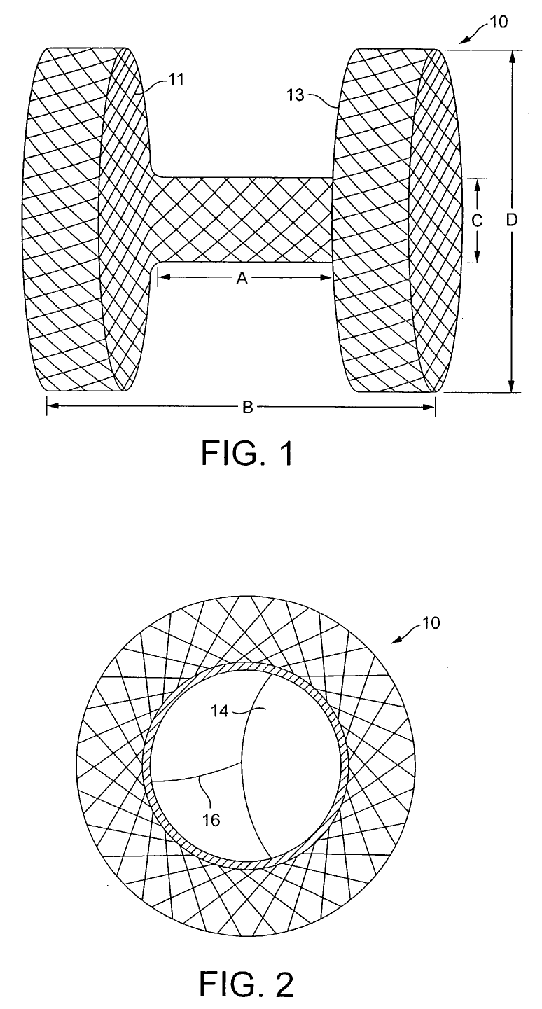

FIG. 1 illustrates a preferred embodiment of an adapter stent 10 according the present invention. It may comprise a woven wire stent fabricated of 0.027 mm diameter Nitinol® wire, heat treated according to conventional techniques to memorize its displayed configuration. The Nitinol® wire employed is chosen to display super-elasticity at room and body temperatures so that it may be compressed for delivery and resume its memorized configuration at the implant site. Other shape memory materials including plastics may be substituted.

In the example illustrated, the adapter stent 10 is a generally tubular structure, defining an interior lumen. It is preferably in the general form of a colo-rectal stent. The adapter stent 10 has enlarged diameter, generally cylindrical proximal and distal portions and a reduced diameter generally cylindrical central portion in which the valved venous segment or other replacement valve is to be mounted. The portions 11 and 13 of the stent between the proximal and distal portions and the central portion generally define radial wall sections, extending from the diameter of the central portion to the diameter of the proximal and distal portions. The inner diameter “C” of the central porion may be about 18 mm, but may be somewhat more or less (e.g. 16-22 mm) depending on the size of the valved venous segment or other replacement valve to be used. The outer diameter “D” of the proximal and distal portions of the stent may be about 30 mm, but again may be somewhat larger or smaller depending on the diameter of the patient's outflow tract. A typical dimension for the overall length “B” of the stent may be about 5.5 cm, with a typical dimension for the middle portion of about 15 mm. Greater or lesser lengths may be employed to, as determined empirically. As discussed below, alternative stent configurations may be employed, as long as they include a smaller diameter portion sized to accept the venous segment or other replacement valve and a larger diameter portion sized to seal against the inner wall of the vessel at the desired implant site.

FIG. 2 is an end view of the adapter stent 10 of FIG. 1, with a valved venous segment 14 installed, illustrating the first embodiment of a replacement valve according to the present invention. Leaflets 16 are visible. The venous segment is sutured to the adapter stent along its proximal and distal edges and preferably is sutured to the stent at most, if not all of the intersections of the wire of the stent which overlie the venous segment. Additional sutures may be employed in the areas between the commissures of the valve. For example, an example of the assembly of suitable valve components is described in more detail in co-pending U.S. Provisional Application, Attorney No. P-0022027.00 filed Nov. 19, 2004.

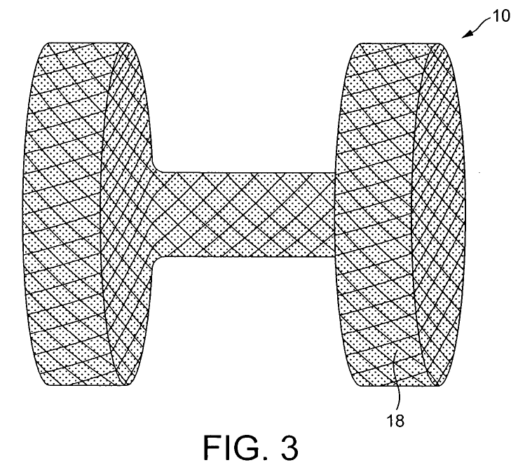

FIG. 3 illustrates the adapter stent of FIG. 1 with a liquid resistant covering 18 applied. This covering may be a 0.3 mm ePTFE membrane of the type presently used to produce covered stents, supplied by Zeus Inc., Orangeburg, S.C. Alternative coverings such as silicone rubber, polyurethane, etc. might also be used. The covering may be a tube or a tape, wound around the stent. The covering may be fastened to the stent using 7.0-propylene thread or adhesives, such as cyanoacrylates. In the context of the invention, it is important that the covering extend over the radial wall portion between the generally cylindrical middle section and the generally cylindrical end sections of the stent, to block fluid flow around the valved venous conduit located in the middle section. Preferably, as illustrated, the covering extends substantially the entire length of the stent so that it will have substantial areas overlying the proximal and distal sections to seal to the vessel wall at the implant site. In the first embodiment of the invention, the covered adapter stent will have the valved venous segment installed as illustrated in FIG. 2. In the second embodiment, the covered adapter stent will be implanted first, without the valved venous segment, as discussed below.

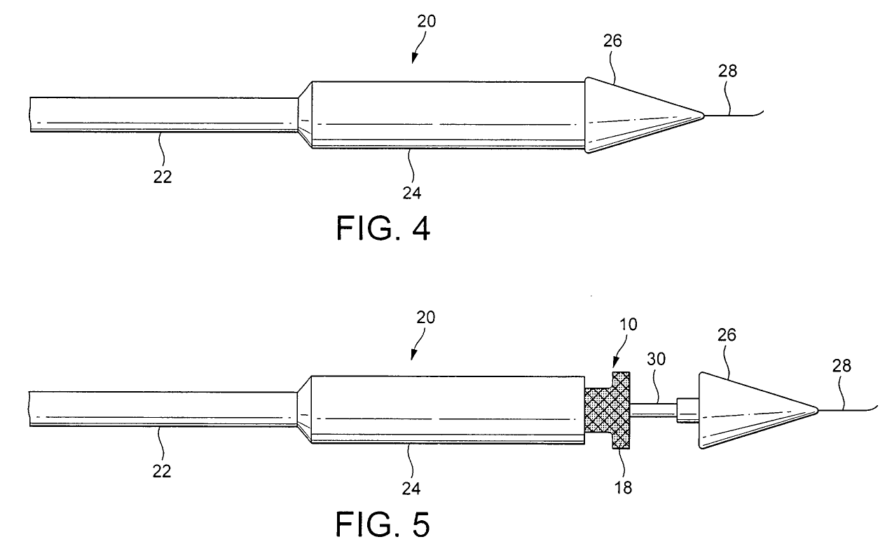

FIG. 4 illustrates a system for delivering a replacement valve according to the first embodiment of the invention and for delivering the adapter stent according to the second embodiment of the invention. The delivery system 20 comprises an outer sheath 22 overlying an inner catheter (not visible in this Figure). The outer sheath has an expanded distal portion 24, within which the adapter stent (with or without valved venous segment) is located. The adapter stent is compressed around the inner catheter and is retained in its compressed configuration by the outer sheath 22. A tapered tip 26 is mounted to the distal end of the inner catheter and serves to ease the passage of the delivery system through the vasculature. The system also includes a guidewire 28, which may be, for example, a 0.089 cm extra stiff guidewire as manufactured by Amplatzer, Golden Valley, Minn. The guidewire is used to guide the delivery system to its desired implant location.

The materials and construction of the delivery system may correspond generally to those described in the above-cited Tower, et al. applications, with the exception that a balloon and balloon inflation lumen are not required. The delivery system is advanced to the desired valve implant site using the guidewire 28, after which the sheath 22 is retracted to allow expansion of the adapter stent. The implant procedures according to both disclosed embodiments of the present invention are also described in the articles: “Percutaneous Pulmonary Valve Replacement in a Large Right Ventricular Outflow Tract”, Boudjemline, et al., Journal of the American College of Cardiology 2004: 43:1082-1087 and “The Year in Congenital Heart Disease”, Graham, Jr., Journal of the American College of Cardiology 2004: 43:2132-2141.

FIG. 5 illustrates the mechanism for deployment of the adapter stent, with or without valved venous segment, at the desired implant site. The outer sheath 22 is moved proximally, allowing the adapter stent 12 to expand away from the inner catheter 30. The distal segment of the adapter stent engages the wall of the heart vessel at the desired implant site, stabilizing the stent. The outer sheath 22 is then moved further proximally, releasing the proximal segment of the adapter stent. The delivery system is then withdrawn proximally. In the first embodiment of the invention, with the valved venous segment pre-mounted, this completes the implant of the replacement valve. In the second embodiment, as described below, the valved venous segment is later inserted into the adapter stent.

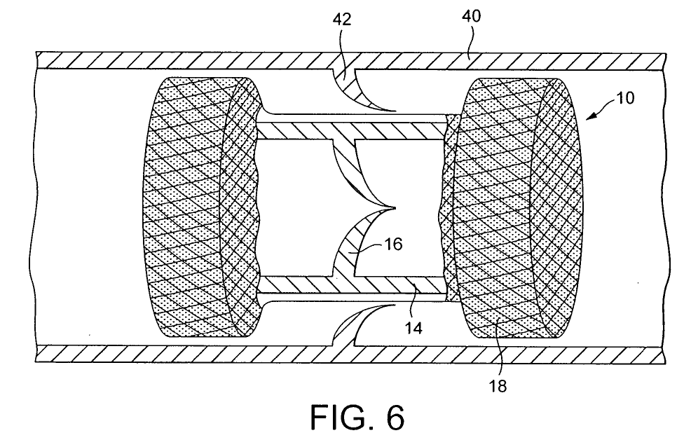

FIG. 6 is a schematic cross section of a replacement valve according to the first embodiment of the invention, as implanted in the right ventricular outflow tract 40. As seen in the Figure, the proximal and distal sections of the adapter stent 10 are expanded against the inner wall of the outflow tract 40. The adapter stent pushes the native valve leaflets 42 aside, allowing implant of the leaflets 16 of the valved venous segment 14 in the original position of the native valve. The adapter stent could also be positioned so that the proximal end segment compresses the native leaflets against the wall of the outflow tract or could also be positioned downstream of the native leaflets. In this Figure, the liquid seal provided by the coating 18 is also illustrated.

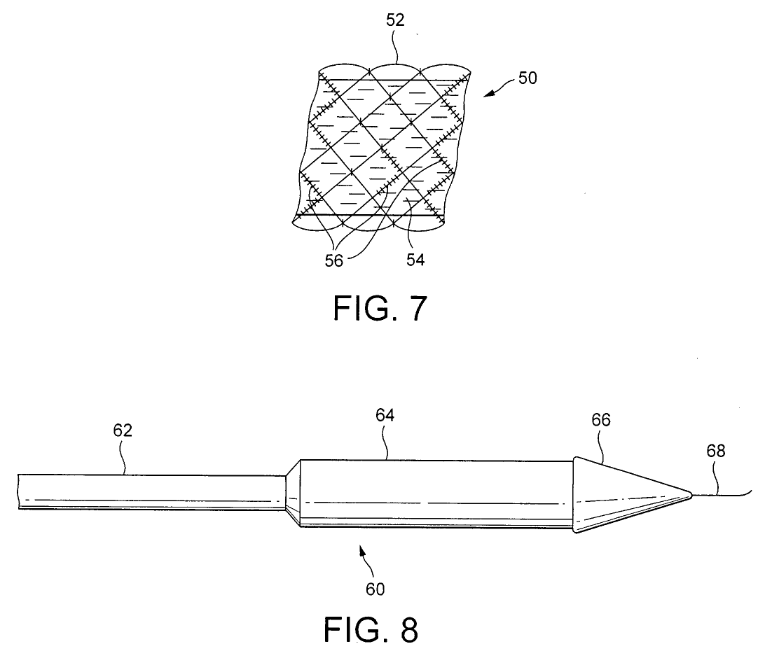

FIG. 7 illustrates a stented valved venous segment 50 which may be used in conjunction with the second embodiment of the invention. The stented venous segment 50 may correspond to that described in the above-cited Tower, et al., and Bonhoeffer et al. references. The stented venous segment is expandable to an outer diameter as large as the inner diameter of middle portion of the adapter stent. The stent 52 may be fabricated of platinum, stainless steel or other biocompatible metal. While it may be fabricated using wire stock as described in the above-cited Tower, et al. applications, it is believed that a more likely commercial embodiment would be produced by machining the stent from a metal tube, as more commonly employed in the manufacture of stents. The specifics of the stent are not critical to the invention, and any known generally cylindrical stent configuration is probably workable. The venous segment 54 is mounted within the stent 52 with its included valve located between the ends of the stent and is secured to the stent it by sutures 56. Sutures 56 are located at the proximal and distal ends of the stent and preferably at all or almost all of the intersections of the stent, as illustrated. A more detailed description of the manufacture of the stented venous segment is disclosed in co-pending U.S. Provisional Application, Attorney No. P-0022027.00 filed Nov. 19, 2004.

FIG. 8 illustrates a system for delivering a valved venous segment as in FIG. 7 to the interior of a previously implanted adapter stent, according to the second embodiment of the invention. The delivery system 60 comprises an outer sheath 62 overlying an inner balloon catheter (not visible in this Figure). The outer sheath has an expanded distal portion 64, within which the stented valved venous segment is located. The venous segment is compressed around a single or double balloon located on the inner catheter. A tapered tip 66 is mounted to the distal end of the inner catheter and serves to ease the passage of the delivery system through the vasculature. The system also includes a guidewire 68, which may be, for example, a 0.089 cm extra stiff guidewire as manufactured by Amplatzer, Golden Valley, Minn. The guidewire is used to guide the delivery system to its desired implant location.

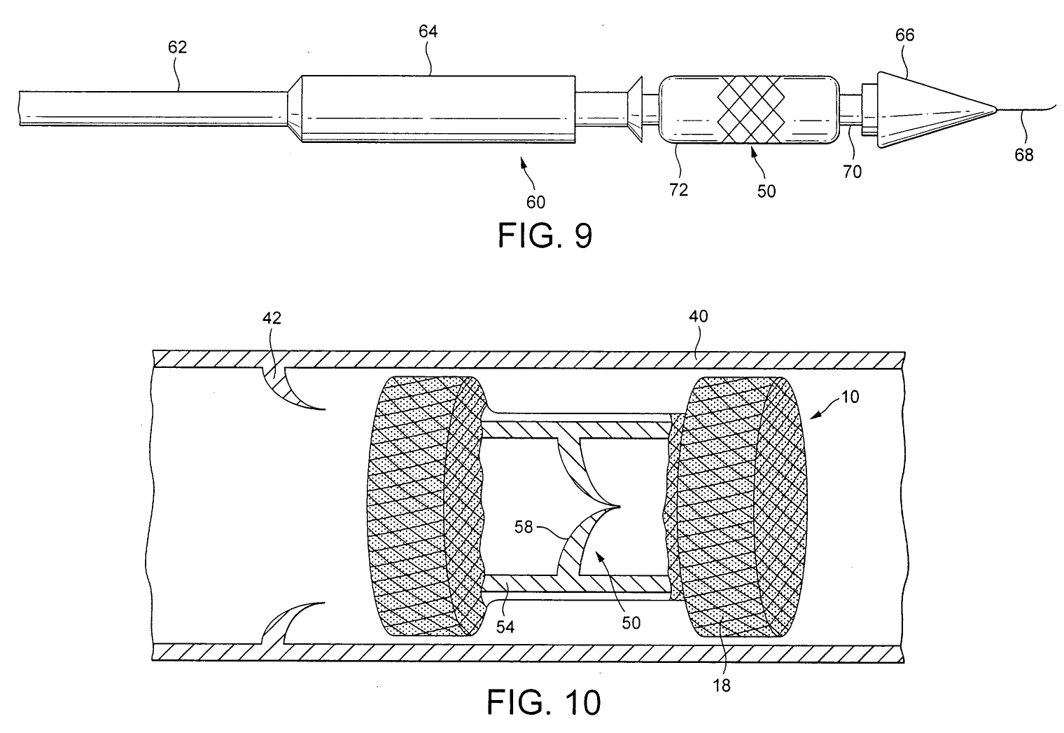

The delivery system and its use may correspond to that described in the above-cited Tower, et al. applications, with the exception that the venous segment is placed within the middle section of a previously placed adapter stent rather than expanded against a failed native or prosthetic valve. The delivery system is advanced to the desired valve implant site using the guidewire 68, after which the sheath 62 is retracted to allow balloon expansion of the venous segment, as illustrated in FIG. 9, discussed below.

FIG. 9 illustrates the mechanism for deployment of the stented valved venous segment 50 within middle portion of a previously implanted adapter stent. The outer sheath 62 is moved proximally, exposing the balloon 72 mounted on inner catheter 70. The balloon 70 is expanded, expanding venous segment 50 against the inner surface of the previously implanted adapter stent, stabilizing and sealing the venous segment within the adapter stent. The balloon is then deflated and the delivery system is withdrawn proximally.

FIG. 10 a schematic cross section of a replacement valve according to the second embodiment of the invention, as implanted in the right ventricular outflow tract 40. As seen in the Figure, the proximal and distal sections of the adapter stent 10 are expanded against the inner wall of the outflow tract 40. The adapter stent in this case is mounted downstream of the native valve leaflets 42, to allow them to continue to function between the time of implant of the adapter stent 10 and the stented venous segment 50. As described in the above-cited Boudjemline, et al. article, using the second embodiment of the invention, the venous segment 50 may be placed within the adapter stent 10 several weeks after its initial implant. In this Figure, the liquid seal provided by the coating 18 is also illustrated. Also visible are the leaflets 58 of venous segment 54.

While the second embodiment of the invention as disclosed relies on the simple expansion of the valve stent 52 against the interior of the adapter stent 10 to secure the valved segment therein, it is believed that in some embodiments of the invention, additional interconnecting mechanisms might be employed,. For example, as disclosed in co-pending U.S. Utility application Ser. No. 10/935,730, filed Sep. 7, 2004, a valve stent having flared ends, or an adapter stent or valve stent provided with hooks, barbs or other interconnecting mechanisms might be employed.

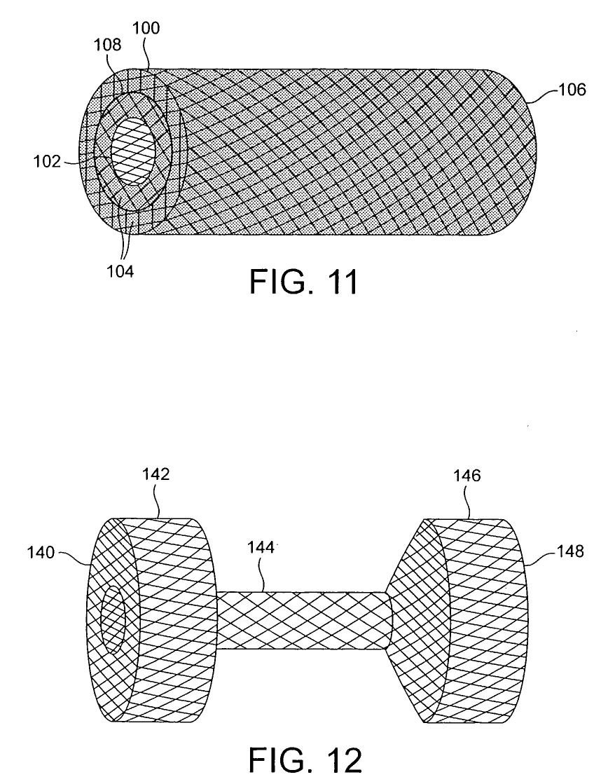

FIG. 11 illustrates a particularly preferred alternative embodiment of an adapter stent for use in conjunction with the present invention. This adapter stent may be employed in conjunction with replacement valves that are mounted to the stent prior to implant of the adapted stent or after implant of the adapter stent, as discussed above. The adapter stent takes the form of a cylindrical toroid, with an inner cylindrical section 102 (an inner portion having a diameter suitable for placement of a valve) in which the replacement valve is mounted, surrounded by an outer, larger diameter cylindrical section 100 (an outer portion having a diameter suitable for contacting the inner walls of a tubular organ). Radial end walls 104 and 106 extend between the inner and outer cylindrical sections. The stent may be made of Nitinol, staring with two woven tubes, nested within one another, the free ends of their wires connected to one another by means of a crimp sleeve 108 at each end and then heat treated to form the structure illustrated. Alternatively, the structure may be formed using a single woven tube of Nitinol wire, defining inner and outer cylindrical sections, the free ends of the wires attached to one another using a crimp sleeve, and the structure thereafter heat treated to form the illustrated configuration. In use, at least the radial walls 104 and 106 are to be provided with a fluid resistant covering.

FIG. 12 illustrates a second alternative embodiment of an adapter stent for use in conjunction with the present invention. This adapter stent may be employed in conjunction with replacement valves that are mounted to the stent prior to implant of the adapted stent or after implant of the adapter stent, as discussed above. This stent takes the general form of a colo-rectal stent formed of a woven Nitinol tube, but with its proximal and distal ends folded back over the central portion of the stent. The stent has a reduced diameter central portion 144 in which the replacement valve is mounted and two larger diameter portions 142 and 146, sized to bear against the wall of the vessel at the desired implant site. Sections 140 and 148 define radial walls. In use, at least the radial walls 140 and 148 are to be provided with a fluid resistant covering.

While the disclosed embodiments employ a self expanding adapter stent, in some embodiments of the invention a balloon expanded adapter stent could be substituted. Likewise, in some versions of the second disclosed embodiment of the invention, a self expanding valve stent might be substituted for the balloon expanded stent described.

Finally, while the invention described above is particularly optimized for placement of valves in the right ventricular outflow tract, it is possible that the invention might be used to place valves in other blood vessels or other tubular organs. Similarly, while bovine jugular veins are disclosed as the source for the valved segments used to practice the invention, other source animals or source vessels may be substituted. Further, alternative replacement valves, for example as described U.S. Pat. Nos. 6,719,789 and 5,480,424, issued to Cox, discussed above. As such, the above description should be taken as exemplary, rather than limiting, in conjunction with the following claims.

All patents, patent applications, publications and journal articles mentioned herein are incorporated herein by reference in their entirety.

1. A method for placing a valve in a tubular organ having a greater diameter than the valve, which method comprises:
delivering an expandable tubular adapter having an outer portion with a diameter suitable for contacting the inner walls of the tubular organ, and an inner portion with a diameter suitable for placement of the valve;
expanding the adapter so that the outer portion contacts the tubular organ; and
placing the valve within the inner portion of the adapter.

2. A method according to claim 1 wherein the tubular organ is a blood vessel and wherein delivering the adapter comprises delivering the adapter to a desired site within the blood vessel and wherein expanding the adapter comprises expanding the adapter so that the outer portion contact the blood vessel.

3. A method according to claim 2 wherein the valve is a segment of bovine jugular vein and the blood vessel is a right ventricular outflow tract and wherein delivering the adapter comprises delivering the adapter to a desired site within the outflow tract and wherein expanding the adapter comprises expanding the adapter so that the outer portion contacts the outflow tract.

4. A method according to claim 2, wherein the adapter comprises a stent and wherein expanding the adapter comprises expanding the stent.

5. A method according to claim 4 wherein the adapter further comprises a liquid resistant covering extending over the outer portion of the adapter and wherein expanding the adapter further comprises expanding the covering to contact the tubular organ.

6. A method according to claim 4, wherein the adapter comprises a self expanding stent and wherein expanding the adapter comprises releasing the adapter from a constraint in order to allow it to self-expand.

7. A method according to claim 4, wherein placing the valve in the adapter occurs prior to expanding the adapter.

8. A method according to claim 4, wherein placing the valve in the adapter occurs after expanding the adapter.

9. A method according to claim 8, wherein the valve is provided with a stent and wherein placing the valve comprises expanding the valve's stent.

10. A method according to claim 9 wherein the stent is a balloon expandable stent and wherein expanding the valve's stent comprises expanding the valve's stent using a balloon.

11. A method of placing a valve in a tubular organ having a greater diameter than the valve, comprising:
delivering an expandable tubular adapter having a larger diameter portion and a lesser diameter portion to a desired site within the tubular organ;
placing the valve within the lesser diameter portion of the adapter.

12. An apparatus for placing a valve in a tubular organ having a greater diameter than the valve, comprising:
an expandable tubular adapter having an outer portion with a diameter suitable for contacting the inner walls of the tubular organ, and an inner portion with a diameter suitable for placement of the valve; a valve mounted within the inner portion of the adapter.

13. An apparatus according to claim 12 wherein the adapter comprises a stent.

14. An apparatus according to claim 13 wherein the adapter further comprises a liquid resistant covering extending over the outer portion of the adapter.

15. An apparatus according to claim 13, wherein the adapter comprises a self expanding stent.

16. An apparatus for placing a valve in a tubular organ having a greater diameter than the valve, comprising:
an expandable tubular adapter having a larger diameter portion and a lesser diameter portion, the larger diameter portion expandable to the diameter of the tubular organ; and
a valve mounted within the lesser diameter of the adapter.

17. A system for placing a valved vascular segment in a tubular organ having a greater inner diameter than the outer diameter of the vascular segment, comprising:
an expandable tubular adapter having an outer portion with a diameter suitable for contacting the inner walls of the tubular organ, and an inner portion with a diameter suitable for placement of the valve;
an expandable valved vascular segment, expandable to the diameter of the inner portion of the adapter.

18. A system according to claim 17 wherein the adapter further comprises a liquid resistant impermeable covering extending over the outer portion of the adapter.

19. A system according to claim 18 wherein the adapter comprises a stent.

20. A system according to claim 19, wherein the adapter comprises a self expanding stent.

21. A system according to claim 17, wherein the valved vascular segment is provided with a stent, expandable to the diameter of the inner portion of the adapter.

22. A system according to claim 21 wherein the vascular segment's stent is a balloon expandable stent.

23. A system for placing a valved vascular segment in a tubular organ having a greater inner diameter than the outer diameter of the vascular segment, comprising:
an expandable tubular adapter having a larger diameter portion and a lesser diameter portion, the larger diameter portion expandable to the inner diameter of the tubular organ; and
an expandable valved vascular segment, expandable to an inner diameter of the lesser diameter portion of the adapter.
